(19) United States
(12) Patent Application Publication
(10) Pub. No.: US 2011/0251540 A1
(43) Pub. Date: Oct. 13, 2011

(54) APPARATUS AND METHOD FOR TREATING PECTUS EXCAVATUM

(76) Inventor: David Notrica, Phoenix, AZ (US)

(21) Appl. No.: 12/759,627

(22) Filed: Apr. 13, 2010

(51) Int. Cl. A61F 5/00

(52) U.S. Cl. 602/19

(57) ABSTRACT

An apparatus and method for treating pectus excavatum are disclosed. A pectus bar stabilizer having two or more channels may be used to secure two or more pectus bars within the chest cavity of a patient. The channels of the pectus bar stabilizer may be spaced apart to support pectus bars that are positioned one intercostal space apart or more than one intercostal space apart.

### FIELD OF THE INVENTION

This invention relates generally to medical devices and, more particularly, to an apparatus and method for treating pectus excavatum.

### BACKGROUND OF THE INVENTION

Pectus excavatum is a congenital chest wall abnormality wherein several ribs and the sternum grow abnormally. As a result, the anterior chest wall appears concave or caved-in.

One method of treating pectus excavatum was developed by Dr. Donald Nuss. The Nuss Technique involves slipping in one or more concave steel bars into the chest, underneath the sternum. The bar is then flipped to a convex position in order to push outward on the sternum, correcting the deformity. Usually the bar stays in the body for about two years, although many surgeons are now moving toward leaving them in for up to five years. When the bones have solidified into place, the bar is removed through outpatient surgery. Many large defects require two bars to be placed to fully correct the deformity.

One major issue with the Nuss Technique, however, is that the bars have a tendency to flip out of position, which may be very painful for the patient. The flip rate may be as high as 15%. The current bar stabilizers used in the Nuss Technique do not seem to prevent this complication. Another drawback of the current bar stabilizers is that it does not prevent the bars from rubbing together when the patient breathes. When this happens, it causes an audible clicking noise. In addition, the current surgery requires a cumbersome procedure to attach the bars to the chest wall around a rib. Still another drawback of the Nuss Technique is that, with repetitive stress, the sternal wires used to affix the stabilizer to the main bar may fracture, erode through the skin, or cause pain requiring reoperation.

### SUMMARY OF THE INVENTION

In accordance with one embodiment of the present invention a pectus bar stabilizer is disclosed. The pectus bar stabilizer comprises a base for securing the pectus bar stabilizer to chest wall muscles of a patient and at least two channels, each channel for receiving a pectus bar therethrough.

In accordance with another embodiment of the present invention, an apparatus for treating pectus excavatum is disclosed. The apparatus comprises at least two convex pectus bars, each pectus bar having a medial portion for weaving underneath a sternum of a patient, a left side lateral portion and a right side lateral portion, each extending from the medial portion in opposing directions, both lateral portions for supporting a pair of ribs of the patient, and a plurality of apertures in each lateral portion for receiving sutures to secure the pectus bar to chest wall muscles of the patient. The apparatus also comprises at least two pectus bar stabilizers, each pectus bar stabilizer having a base for securing the pectus bar stabilizer to the chest wall muscles of the patient and at least two channels, each channel for receiving the lateral portion of a pectus bar therethrough.

In accordance with another embodiment of the present invention, a method for treating pectus excavatum is disclosed. The method comprises the steps of providing a first pectus bar and at least a second pectus bar, providing a first pectus bar stabilizer and a second pectus bar stabilizer, positioning the first pectus bar and the second pectus bar within a chest cavity of a patient, securing the first pectus bar and the second pectus bar to the first pectus bar stabilizer, securing the first pectus bar and the second pectus bar to the second pectus bar stabilizer, securing the first pectus bar and the second pectus bar to chest wall muscles of the patient, and securing the first pectus bar stabilizer and the second pectus bar stabilizer to the chest wall muscles of the patient.

The foregoing and other objects, features, and advantages of the invention will be apparent from the following, more particular description of the preferred embodiments of the invention, as illustrated in the accompanying drawings.

### BRIEF DESCRIPTION OF THE DRAWINGS

The present invention will become more fully understood from the detailed description and the accompanying drawings, wherein:

FIG. 1 is an elevated perspective view of an apparatus for treating pectus excavatum in accordance with the present invention.

FIG. 1A is an elevated perspective view of another embodiment of an apparatus for treating pectus excavatum in accordance with the present invention.

FIG. 2A is a perspective view of the apparatus of FIG. 1 shown in use and attached in front of the sternum and ribs.

FIG. 2B is a top view of the apparatus of FIG. 1 shown in use and attached behind the sternum and ribs.

FIG. 3 is a cross-sectional view of the pectus bar stabilizer of the apparatus of FIG. 1.

FIG. 4 is a cross-sectional view of the pectus bar stabilizer of the apparatus of FIG. 1 shown with a screw being inserted to secure the pectus bar to the pectus bar stabilizer.

FIG. 5 is a cross-sectional view of the pectus bar stabilizer of the apparatus of FIG. 1 shown with a screw being used to secure the pectus bar to the pectus bar stabilizer.

FIG. 6 is a top view of the apparatus of FIG. 1.

FIG. 7 is a top view of the apparatus of FIG. 1A.

### DETAILED DESCRIPTION OF THE PREFERRED EMBODIMENTS

The novel features believed characteristic of the invention are set forth in the appended claims. The invention will best be understood by reference to the following detailed description of illustrated embodiments when read in conjunction with the accompanying drawings, wherein like reference numerals and symbols represent like elements.

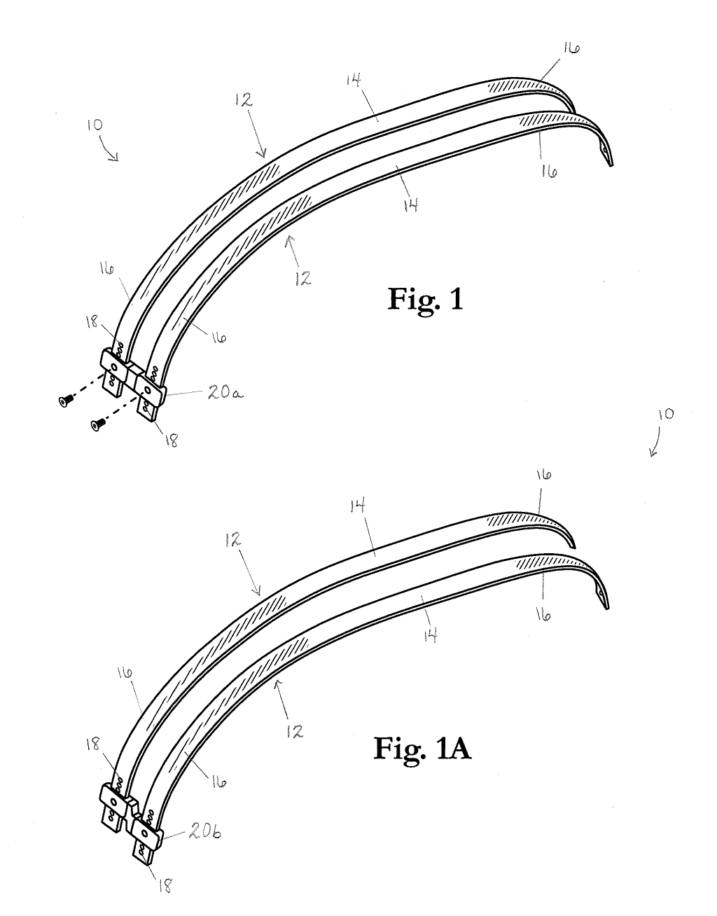

Referring to FIGS. 1-7, an apparatus for treating pectus excavatum, hereinafter apparatus 10, and a related method are shown. In its simplest form, the apparatus 10 comprises a pectus bar stabilizer 20 for securing at least two pectus bars 12 in place within the chest cavity of a patient.

FIGS. 1 and 1A show two pectus bars 12 secured with a pectus bar stabilizer 20. FIG. 1 shows one embodiment of the pectus bar stabilizer 20a (referred to generally as pectus bar stabilizer 20) and FIG. 1A shows another embodiment of the pectus bar stabilizer 20b (referred to generally as pectus bar stabilizer 20). The pectus bars 12 are convex and each have a medial portion 14 and two lateral portions 16 that extend in opposite directions from the medial portion 14. The lateral portions 16 together support a pair of the patient's ribs 100. In one embodiment, the lateral portions 16 may have a plurality of apertures 18. These apertures 18 may be used for receiving sutures that secure the pectus bars 12 to the chest wall muscles of the patient; the apertures 18 may also be used for securing the pectus bars 12 to the pectus bar stabilizer 20 with securing devices 36; or for both purposes.

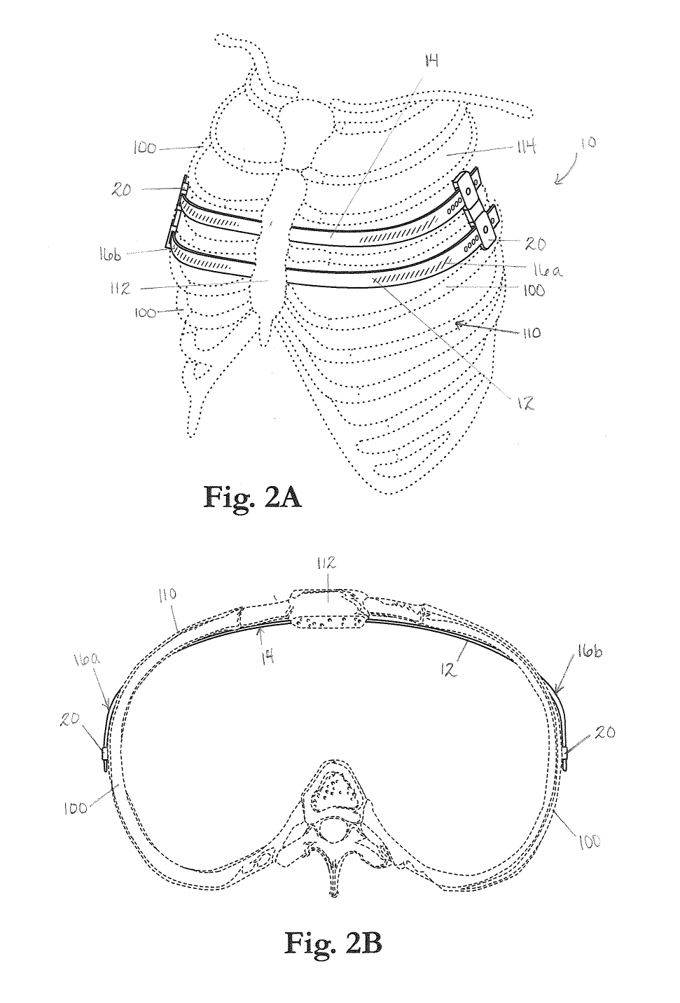

FIGS. 2A and 2B show how the apparatus 10 will be placed within the chest cavity of the patient. Typically, each pectus bar 12 is pulled under the sternum 112 from the patient's side with the convexity facing posteriorly. When the pectus bars 12 are in position, they are flipped, causing the sternum 112 and the anterior chest wall to rise into the proper position. The lateral portions 16 of each pectus bar 12 then rests on the anterior surface 110 of a pair of the patient's ribs 100. One pectus bar stabilizer 20 is coupled to the left side lateral portion 16a (referred to generally as lateral portion 16) of each of the pectus bars 12. Then another pectus bar stabilizer 20 is coupled to the right side lateral portion 16b (referred to generally as lateral portion 16) of each of the pectus bars 12.

FIG. 2A shows that two pectus bars 12 being used with the apparatus 10, however, it should be clearly understood that substantial benefit may be derived from the pectus bar stabilizer 20 being constructed to secure more than two pectus bars 12. For example, the pectus bar stabilizer 20 could have three channels 26 to secure three pectus bars 12. FIG. 2A also shows that the pectus bars 12 are positioned only one intercostal space 114 apart. It should also be clearly understood that further substantial benefit may be derived from the pectus bar stabilizer 20 being constructed to secure pectus bars 12 that are positioned more than one intercostal space 114 apart.

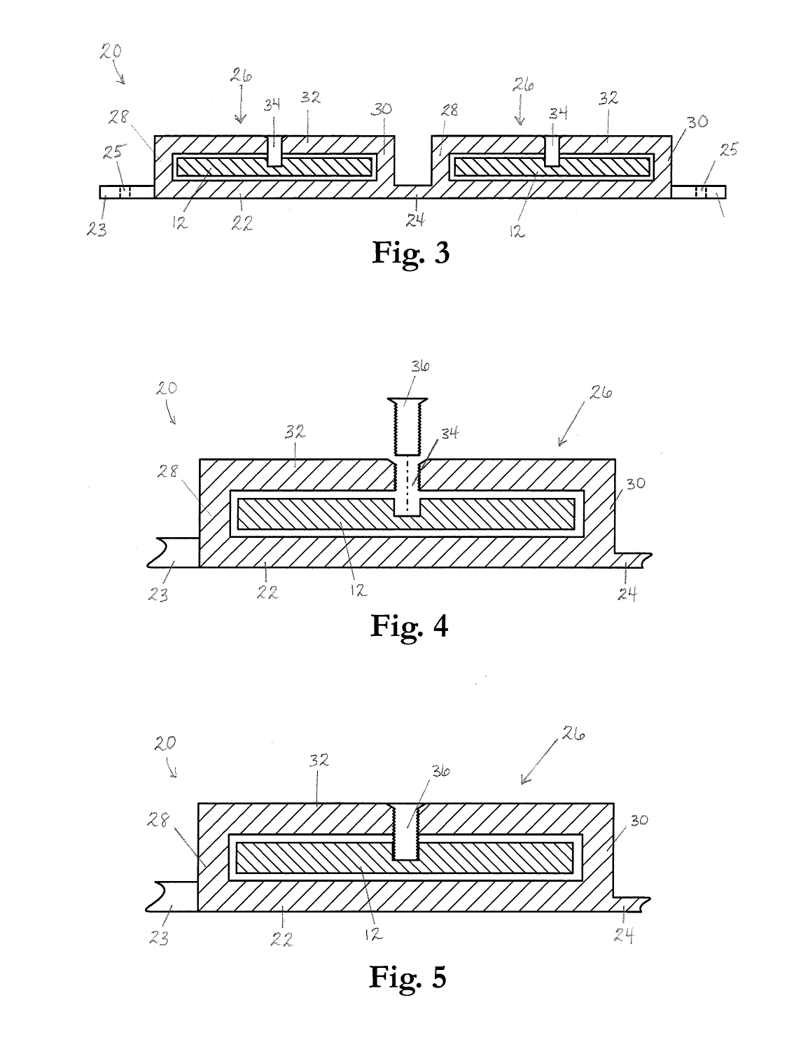

FIGS. 3-5 show a pectus bar stabilizer 20. The pectus bar stabilizer 20 has a base 22 and at least two channels 26. In one embodiment, each channel 26 fully encloses the pectus bar 12 that is inserted into it. In one embodiment, the base 22 has a middle portion 24 between two channels 26. In the figures, the middle portion 24 is shown to bridge one intercostal space 114 between two ribs 100 of the patient, but it should be clearly understood that the middle portion 24 may be longer in order to bridge more than one intercostal space 114. The middle portion 24 may form an open channel between the first end 28 of one channel 26 and the second end 30 of the adjacent channel 26, as shown in FIG. 3. Alternatively, the middle portion 24 may be solid and level with the top surfaces 32 of the two channels 26.

The base 22 may also have apertures 25 in each end 23 of the base 22 which may be used to secure the pectus bar stabilizer 20 to the chest wall muscles of the patient. Each channel may be have a first end 28 extending perpendicularly from the base 22, a second end 30 also extending perpendicularly from the base 22, and a top surface 32 that joins the first end 28 to the second end 30 and that is parallel to the base 22. The middle portion 24 may be situated between the first end 28 of one channel 26 and the second end 30 of an adjacent channel 26. The pectus bar stabilizer 20 may also have an aperture 34 in the top surface 32 of each channel 26. The aperture 34 in the top surface 32 of a channel 26 may be aligned with one of the apertures 18 in the lateral portion 16 of one of the pectus bars 12. A securing device 36 may then be inserted through the aligned apertures 34/18 to secure each pectus bar 12 to the pectus bar stabilizer 20. In one embodiment, the apertures 18 in the lateral portions 16 of the pectus bars 12 and the apertures 34 in the channels 26 of the pectus bar stabilizers 20 may be threaded and a threaded securing device 36 (such as a screw) may be used to secure the pectus bars 12 to the pectus bar stabilizers 20.

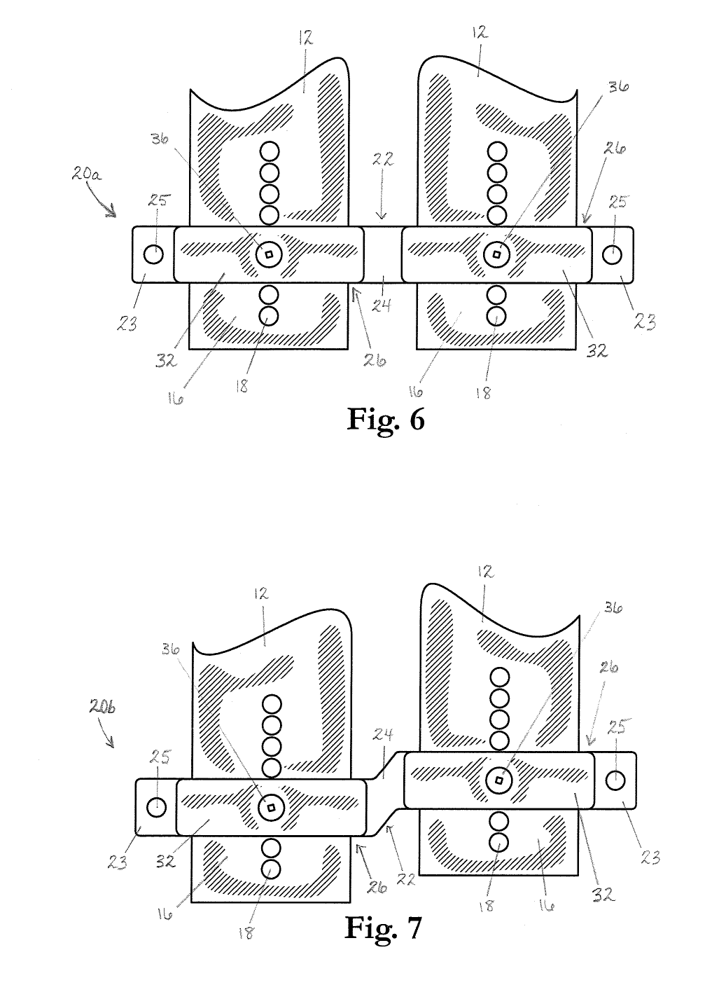

FIG. 6 shows one embodiment of the pectus bar stabilizer 20a. In this embodiment, the middle portion 24 of the base 22 is straight so that the channels 26 are aligned with each other.

FIG. 7 shows another embodiment of the pectus bar stabilizer 20b. The middle portion 24 of the base 22 in this pectus bar stabilizer 20b is angled so that the two channels 26 are offset from one another. Having an angled middle portion 24 allows the pectus bar stabilizer 20b to be to be positioned onto the pectus bars 12 more easily.

### STATEMENT OF USE

Referring to 2A, a method of treating pectus excavatum is disclosed. One pectus bar 12 is pulled under the sternum 112 from the patient's side with the convexity facing posteriorly. A second pectus bar 12 is then also pulled under the sternum 112 in the same manner. It should be clearly understood that while only two pectus bars 12 are shown being used, substantial benefit may be obtained from the use of more than two pectus bars 12. When the pectus bars 12 are in position, they are flipped, causing the sternum 112 and the anterior chest wall to rise into the proper position. The lateral portions 16 of each pectus bar 12 then rests on the anterior surface 110 of a pair of the patient's ribs 100. While it is shown that one pectus bar 12 is positioned one intercostal space 114 above the other pectus bar 12, it should be clearly understood that further substantial benefit may be derived from the pectus bars 12 being positioned more than one intercostal space 114 apart.

The left side lateral portion 16a of the first pectus bar 12 is inserted into the first channel 26 of the first pectus bar stabilizer 20 and the left side lateral portion 16a of the second pectus bar 12 is inserted into the second channel 26 of the first pectus bar stabilizer 20. The right side lateral portion 16b of the first pectus bar 12 is inserted into the first channel 26 of the second pectus bar stabilizer 20 and the right side lateral portion 16a of the second pectus bar 12 is inserted into the second channel 26 of the second pectus bar stabilizer 20.

The apertures 34 in the top surfaces 32 of the channels 26 of the first pectus bar stabilizer 20 are then aligned with the apertures 18 in the left side lateral portions 16a of the pectus bars 12. A securing device 36 is then inserted into the aligned apertures 18/34 to secure the pectus bars 12 to the first pectus bar stabilizer 20. The apertures 34 in the top surfaces 32 of the channels 26 of the second pectus bar stabilizer 20 are also then aligned with the apertures 18 in the right side lateral portions 16b of the pectus bars 12. A securing device 36 is then inserted into the aligned apertures 18/34 to secure the pectus bars 12 to the second pectus bar stabilizer 20.

Sutures may then be inserted through the apertures 25 in the ends 23 of the base 22 to secure the pectus bar stabilizer 20 to the chest wall muscles of the patient. Sutures may also be inserted through the apertures 18 in the lateral portions 16 of the pectus bars 12 to secure the pectus bars 12 to the chest wall muscles of the patient.

While the invention has been particularly shown and described with reference to preferred embodiments thereof, it will be understood by those skilled in the art that the foregoing and other changes in form and details may be made therein without departing from the spirit and scope of the invention.

What is claimed is:

1. A pectus bar stabilizer comprising:
a base for securing the pectus bar stabilizer to chest wall muscles of a patient; and
at least two channels, each channel for receiving a pectus bar therethrough.

2. The pectus bar stabilizer of claim 1 wherein each channel comprises:
a first end extending perpendicularly from the base;
a second end extending perpendicularly from the base; and
a top surface that joins the first end to the second end and that is parallel to the base.

3. The pectus bar stabilizer of claim 2 further comprising an aperture in the top surface of each channel for receiving a securing device.

4. The pectus bar stabilizer of claim 1 wherein the base further comprises a middle portion between the at least two channels and wherein the middle portion bridges at least one intercostal space between two ribs of the patient.

5. The pectus bar stabilizer of claim 4 wherein the middle portion is angled so that the two channels are offset from one another.

6. The pectus bar stabilizer of claim 1 further comprising at least one aperture at each end of the base, each aperture for receiving sutures to secure the pectus bar stabilizer to the chest wall muscles of the patient.

7. The pectus bar stabilizer of claim 1 further comprising at least two pectus bars, each pectus bar being convex and comprising:
a medial portion for weaving underneath a sternum of the patient;
two lateral portions extending in opposing directions from the medial portion, the lateral portions for resting on an anterior portion of a rib of the patient; and
a plurality of apertures in each lateral portion for at least one of receiving sutures to secure the pectus bar to the chest wall muscles of the patient and securing the pectus bar to the pectus bar stabilizer;
wherein the lateral portion of each pectus bar is inserted through one of the channels of the pectus bar stabilizer; and
wherein one pectus bar is placed at least one intercostal space apart from another pectus bar.

8. An apparatus for treating pectus excavatum comprising:
at least two convex pectus bars, each pectus bar having:
a medial portion for weaving underneath a sternum of a patient;
a left side lateral portion and a right side lateral portion, each extending from the medial portion in opposing directions, both lateral portions for supporting a pair of ribs of the patient; and
a plurality of apertures in each lateral portion;
at least two pectus bar stabilizers, each pectus bar stabilizer having:
a base for securing the pectus bar stabilizer to the chest wall muscles of the patient; and
at least two channels, each channel for receiving the lateral portion of a pectus bar therethrough.

9. The apparatus of claim 8 wherein each pectus bar stabilizer further comprises:
a first channel having:
a first end extending perpendicularly from the base;
a second end extending perpendicularly from the base;
a top surface that joins the first end to the second end and that is parallel to the base; and
an aperture in the top surface for aligning with one of the apertures in the lateral portion of one of the pectus bars and for receiving a securing device therethrough;
at least a second channel having:
a first end extending perpendicularly from the base;
a second end extending perpendicularly from the base;
a top surface that joins the first end to the second end and that is parallel to the base; and
an aperture in the top surface for aligning with one of the apertures in the lateral portion of at least a second pectus bar and for receiving a securing device therethrough.

10. The apparatus of claim 8 wherein the pectus bar stabilizer further comprises a middle portion between the two channels and wherein the middle portion bridges at least one intercostal space between two ribs of the patient.

11. The apparatus of claim 10 wherein the middle portion of the pectus bar stabilizer is angled so that the two channels are offset from one another.

12. The apparatus of claim 8 wherein the pectus bar stabilizer further comprises at least one aperture at each end of the base, each aperture for receiving sutures to secure the pectus bar stabilizer to the chest wall muscles of the patient.

13. A method for treating pectus excavatum comprising the steps of:
providing a first pectus bar and at least a second pectus bar;
providing a first pectus bar stabilizer and a second pectus bar stabilizer;
positioning the first pectus bar and the second pectus bar within a chest cavity of a patient;
securing the first pectus bar and the second pectus bar to the first pectus bar stabilizer;
securing the first pectus bar and the second pectus bar to the second pectus bar stabilizer;
securing the first pectus bar and the second pectus bar to chest wall muscles of the patient; and
securing the first pectus bar stabilizer and the second pectus bar stabilizer to the chest wall muscles of the patient.

14. The method of claim 13 wherein each pectus bar comprises:
a medial portion for weaving underneath a sternum of a patient;
a left side lateral portion and right side lateral portion, each extending from the medial portion in opposing directions, both lateral portions for supporting a pair of ribs of the patient; and
a plurality of apertures in each lateral portion.

15. The method of claim 14 wherein each pectus bar stabilizer comprises:
a base for securing the pectus bar stabilizer to the chest wall muscles of the patient;
a first channel having:
a first end extending perpendicularly from the base;
a second end extending perpendicularly from the base;
a top surface that joins the first end to the second end and that is parallel to the base; and
an aperture in the top surface for aligning with one of the apertures in one of the lateral portions of the first pectus bar and for receiving a securing device therethrough;
at least a second channel having:
a first end extending perpendicularly from the base;
a second end extending perpendicularly from the base;
a top surface that joins the first end to the second end and that is parallel to the base; and
an aperture in the top surface for aligning with one of the apertures in one of the lateral portions of the second pectus bar and for receiving a securing device therethrough.

16. The method of claim 15 wherein each pectus bar stabilizer further comprises a middle portion between the first channel and the second channel and wherein the middle portion bridges at least one intercostal space between two ribs of the patient.

17. The method of claim 16 wherein each pectus bar stabilizer further comprises a middle portion between the two channels and wherein the middle portion is angled so that the first channel and the at the second channel are offset from one another.

18. The method of claim 15 wherein each pectus bar stabilizer further comprises at least one aperture at each end of the base, each aperture for receiving sutures to secure the pectus bar stabilizer to the chest wall muscles of the patient.

19. The method of claim 15 further comprising the steps of:
inserting the left side lateral portion of the first pectus bar through the first channel of the first pectus bar stabilizer;
inserting the left side lateral portion of the second pectus bar through the second channel of the first pectus bar stabilizer;
aligning the aperture in the top surface of the first channel of the first pectus bar stabilizer with one of the apertures in the left side lateral portion of the first pectus bar;
aligning the aperture in the top surface of the second channel of the first pectus bar stabilizer with one of the apertures in the left side lateral portion of the second pectus bar;
inserting the right side lateral portion of the first pectus bar through the first channel of the second pectus bar stabilizer;
inserting the right side lateral portion of the second pectus bar through the second channel of the second pectus bar stabilizer;
aligning the aperture in the top surface of the first channel of the second pectus bar stabilizer with one of the apertures in the right side lateral portion of the first pectus bar;
aligning the aperture in the top surface of the second channel of the second pectus bar stabilizer with one of the apertures in the right side lateral portion of the second pectus bar; and
inserting a securing device through each of the aligned apertures.

20. The method of claim 19 wherein the apertures and the securing devices are threaded.
